(19) United States
(12) Patent Application Publication
(10) Pub. No.: US 2025/0064593 A1
(43) Pub. Date: Feb. 27, 2025

(54) GLENOSPHERE ASSEMBLY

(71) Applicant: HOWMEDICA OSTEONICS CORP., Mahwah, NJ (US)

(72) Inventors: Benjamin DASSONVILLE, Plateau des petites roches (FR); Gilles HENRY, Le Pont de Claix (FR); Olivier ZANARDI, Bernin (FR)

(73) Assignee: HOWMEDICA OSTEONICS CORP., Mahwah, NJ (US)

(21) Appl. No.: 18/847,332

(22) PCT Filed: Mar. 27, 2023
(86) PCT No.: PCT/US2023/064983

§ 371 Date: Sep. 16, 2024

Related U.S. Application Data
(60) Provisional application No. 63/362,984, filed on Apr. 14, 2022.

(51) Int. Cl. A61F 2/40; A61F 2/30

(52) U.S. Cl. CPC A61F 2/4081; A61F 2002/30331; A61F 2002/30476; A61F 2002/30507; A61F 2002/4085; A61F 2210/00

(57) ABSTRACT

Provided herein are various embodiments of an improved glenoid implant assembly that includes a baseplate, a polymer glenosphere, and an interfacing component. The interfacing component can be made of a material that is more rigid than the polymer glenosphere, has a bowl-shaped configuration with a convex side and a recessed side, where the convex side is configured to be secured to the circular recessed portion of the polymer glenosphere and the recessed side is configured to securely receive the baseplate. The interfacing component is interposed between the polymer glenosphere and the baseplate and provide a secure attachment between the polymer glenosphere and the baseplate.

### CROSS-REFERENCE TO RELATED APPLICATIONS

This application claims priority under 35 U.S.C. § 119 (e) to U.S. Provisional Application No. 63/362,984, filed on Apr. 14, 2022, the entire contents of which are incorporated herein by reference.

### FIELD OF DISCLOSURE

The present disclosure generally relates to glenoid implants for shoulder prosthesis and more specifically glenoid implants configured with polymer glenosphere.

### BACKGROUND

A shoulder prosthesis includes a glenoid implant intended to replace the glenoid cavity of the scapula and/or a humeral implant intended to replace the humeral head. The glenoid implant generally includes an articular body intended to articulate with the humeral head, and a fixation means to stabilize the articular body with respect to the scapula.

Currently, clinical literature shows a high rate of radiolucency around glenoid cemented, non-cemented, and hybrid components in long-term use of those glenoid implants. One issue is the potential for rocking of the implant in the superior-inferior direction. Currently, cemented implants provide good short- and mid-term fixation, but loosen over time. The current hybrid cemented press-fit implants show similar performance. Press-fit implants show high loosening at mid- to long-term time points.

One of the challenges with the conventional press-fit glenoid implants is that it is difficult to reliably secure the implants to the bone. As a result, hybrid cemented press-fit implants have increased in popularity. Adding a modular metal tray with screws is a solution that is used in many other joints, however in the shoulder, there is often insufficient space for a modular tray. In addition, the modular metal trays are often more stiff than desired.

Thus, improved glenoid implant design that offers enhanced and durable primary fixation to the bone is desired.

### SUMMARY

Provided herein are various embodiments of an improved glenoid implant assembly that includes a polymer glenosphere.

A glenoid assembly according to some embodiments comprises: a baseplate that comprises a proximal end, a distal end, an outer periphery, and a frustoconical side surface extending from the proximal end to the distal end and defining a male-type tapered surface and configured to be secured to a glenoid; a polymer glenosphere having a proximal end and a distal end, including a convex articular surface extending distally from the proximal end and a distal surface provided on the distal end, and a circular recessed portion recessed from the distal surface and forming an interior contour of the circular recessed portion; and an interfacing component, made of a material that is more rigid than the polymer glenosphere, having a bowl-shaped configuration with a convex side and a recessed side, wherein the convex side is configured to secure to the circular recessed portion of the polymer glenosphere; where the recessed side of the interfacing component is configured with an annular interior surface defining a female-type tapered surface to receive and form a friction lock engagement with the male-type tapered surface of the baseplate, and where when the polymer glenosphere, the interfacing component, and the baseplate are assembled into the glenoid implant assembly, the interfacing component is interposed between the polymer glenosphere and the baseplate and provide a secure attachment between the polymer glenosphere and the baseplate.

Also disclosed is a glenoid implant assembly that comprises: a polymer glenosphere having a proximal end and a distal end, including a convex articular surface extending distally from the proximal end and a distal surface provided on the distal end, and a circular recessed portion recessed from the distal surface and forming an interior contour of the circular recessed portion; and an interfacing component, made of a material that is more rigid than the polymer glenosphere, having a bowl-shaped configuration with a convex side and a recessed side, wherein the convex side is configured to secure to the circular recessed portion of the polymer glenosphere; where the recessed side of the interfacing component is configured with an annular interior surface defining a female-type tapered surface to receive and form a friction lock engagement with the male-type tapered surface of the baseplate, and where when the polymer glenosphere and the interfacing component are assembled, the interfacing component provides a rigid frame for the polymer glenosphere.

### BRIEF DESCRIPTION OF THE DRAWINGS

The various embodiments of the inventive glenoid implant of the present disclosure will be described in more detail in conjunction with the following drawing figures. The structures in the drawing figures are illustrated schematically and are not necessarily intended to show actual dimensions or relative scale.

FIG. 1 is an illustration showing a longitudinal cross-section of a glenoid implant assembly according to the present disclosure.

FIG. 2 is an illustration showing a polymer glenosphere component of the glenoid implant assembly according to the present disclosure.

FIG. 3 is an illustration showing a securing screw component of the glenoid implant assembly according to the present disclosure.

FIG. 4 is an illustration showing an interfacing component of the glenoid implant assembly according to the present disclosure.

FIG. 5A is an illustration showing a tightening nut component of the glenoid assembly according to the present disclosure.

FIG. 5B is an illustration showing a longitudinal cross-sectional view of the tightening nut component shown in FIG. 5A.

FIG. 6 is an illustration of a plan view of the polymer glenosphere component of the glenoid implant assembly viewed from its proximal end.

FIG. 7 is an illustration of a plan view of an assembly of the polymer glenosphere component, the interfacing component, and the tightening nut viewed from the distal end.

FIG. 8A is an illustration of a lateral view of an assembly of the polymer glenosphere component, the interfacing component, the tightening nut, and the securing screw.

FIG. 8B is an illustration of a longitudinal cross-sectional view of the assembly of FIG. 8A.

FIG. 8C is a detailed view of the area A identified in FIG. 8B.

FIGS. 9A-9B are illustrations showing the baseplate component of the glenoid implant assembly according to the present disclosure.

### DETAILED DESCRIPTION

This description of the exemplary embodiments is intended to be read in connection with the accompanying drawings, which are to be considered part of the entire written description. The drawing figures are not necessarily to scale and certain features may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. When only a single machine is illustrated, the term “machine” shall also be taken to include any collection of machines that individually or jointly execute a set (or multiple sets) of instructions to perform any one or more of the methodologies discussed herein. The term “operatively connected” is such an attachment, coupling or connection that allows the pertinent structures to operate as intended by virtue of that relationship. In the claims, means-plus-function clauses, if used, are intended to cover the structures described, suggested, or rendered obvious by the written description or drawings for performing the recited function, including not only structural equivalents but also equivalent structures.

Any references to “a humeral head” as used herein should be construed to include both an anatomical humeral head as well as implant humeral head.

Provided herein are various embodiments of an improved glenoid implant in reverse configuration that incorporates polymer glenosphere and a new mechanism for securing the polymer glenosphere to a baseplate.

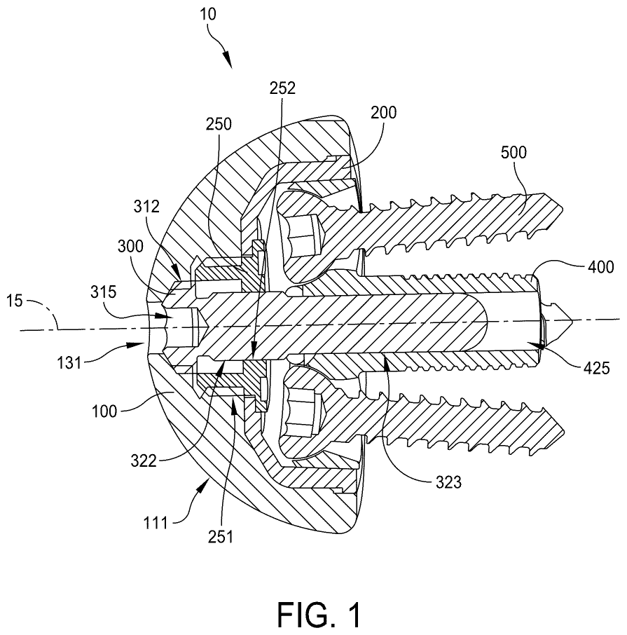

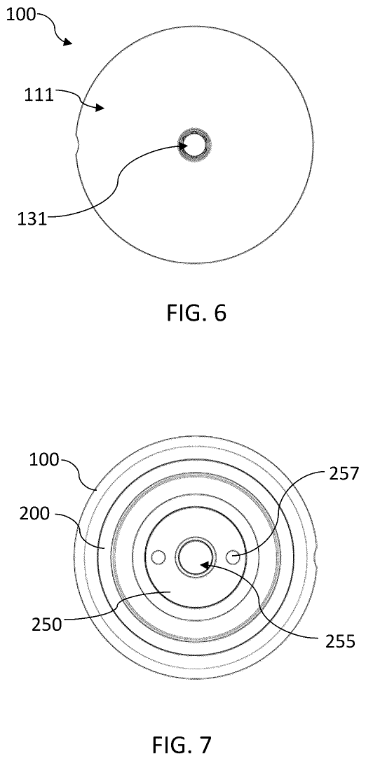

Referring to FIGS. 1-9B, according to some embodiments, an improved glenoid implant assembly 10 comprises a polymer glenosphere 100, an interfacing component 200, and a baseplate 400.

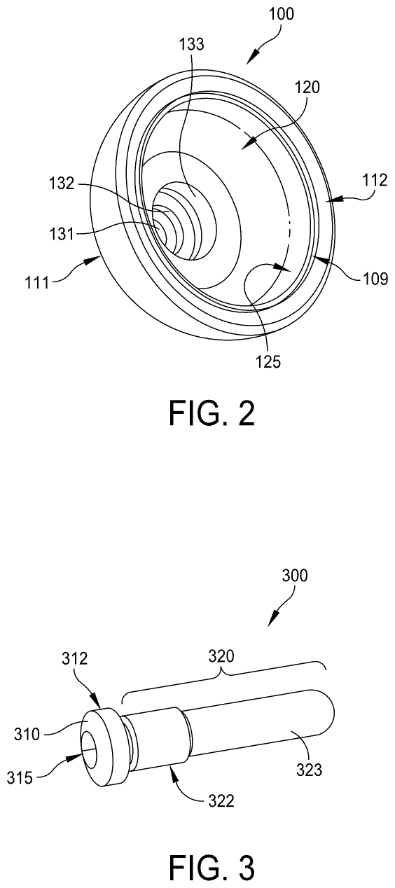

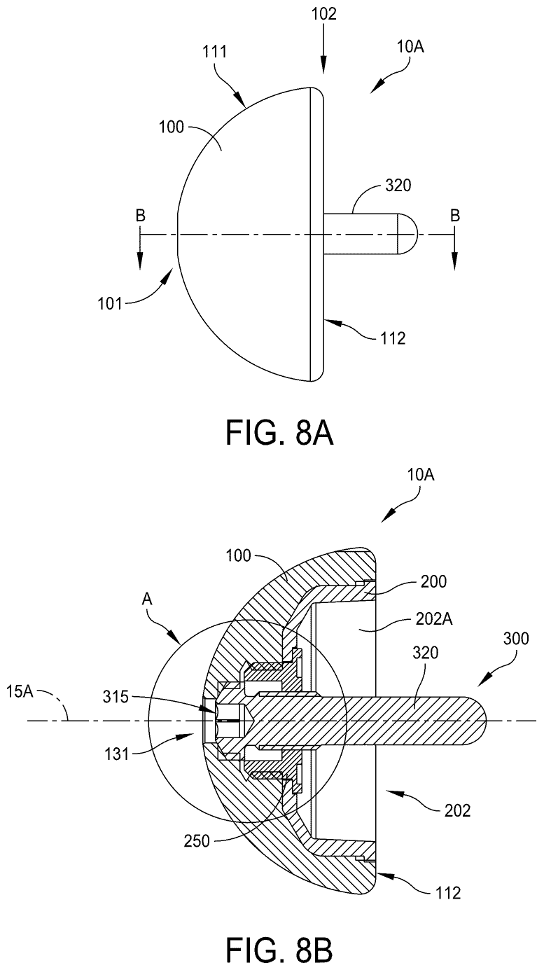

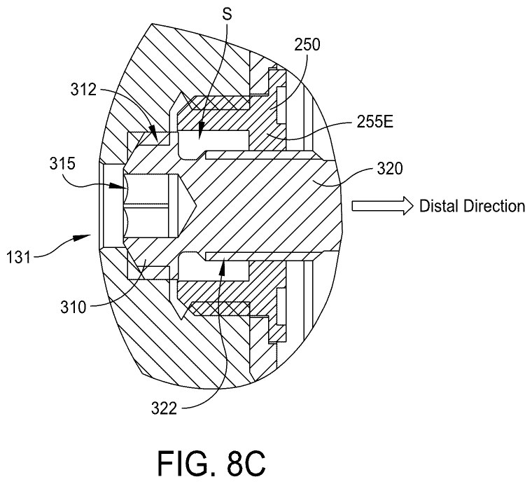

Referring to FIG. 1, the polymer glenosphere 100 is configured to be secured to a glenoid via cooperation of the interfacing component 200 and the baseplate 400. Referring to FIGS. 2, 8A, and 8B, the polymer glenosphere 100 comprises a proximal end 101 and a distal end 102, including an outer surface of the glenosphere 100 convexly contoured to form a convex articular surface 111, a distal surface 112, and a circular recessed portion 120. The convex articular surface 111 extends radially outward, with respect to the longitudinal axis 15A, from the proximal end 101 toward the distal end 102. The convex articular surface 111 can be contoured as a spherical surface or a non-spherical surface. The convex articular surface 111 forms substantially a half of a sphere or a portion of a sphere. This can be better seen in the side view shown in FIG. 8A.

The distal surface 112 is at the distal end 102 of the glenosphere 100. As can be seen in FIG. 2, the circular recessed portion 120 is recessed from the distal surface 112 toward the proximal end 101. The recessed portion 120 can have an interior contour that includes an annular sidewall 125.

In some embodiments of the glenoid implant, the polymer glenosphere 100 can be made of polymers such as polyether ether ketone (PEEK), high-modulus polyethylene (HMPE), and ultra-high-molecular-weight polyethylene (UHMWPE), etc. that are selected to provide the desired performance for the glenosphere. All references to UHMWPE herein include all variants of UHMWPE in orthopedic application such as vitamin E diffused UHMWPE.

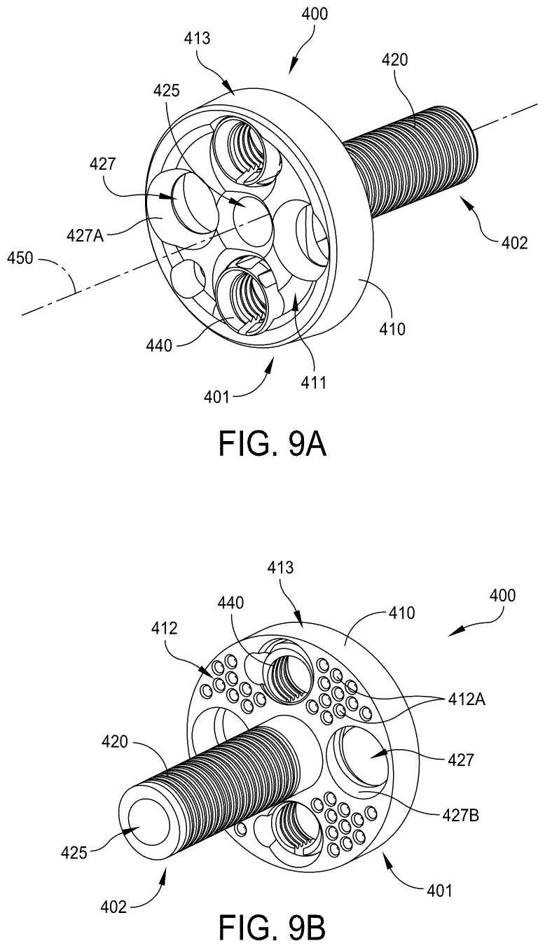

Referring to FIGS. 1, 9A, and 9B, the baseplate 400 comprises a proximal end 401, a distal end 402, a generally disc-shaped body 410, and a central stem 420 extending from the body 410 toward the distal end 402. The body 410 comprises a proximal surface 411, a distal surface 412, and a frustoconical side surface 413 extending between the proximal surface 411 and the distal surface 412. The distal surface 412 is the bone-facing surface that would contact the glenoid when the baseplate 400 is implanted in a patient's shoulder. The distal surface 412 can be provided with a plurality of dimpled features 412A that can promote boney tissue ingrowth after the baseplate 400 is implanted onto a glenoid. The central stem 420 extends from the distal surface 412 along the baseplate's longitudinal axis 450.

The frustoconical side surface 413 defines a male-type tapered surface (e.g. Morse taper) that is configured to secure to the interfacing component 200 by establishing a friction lock. The frustoconical side surface 413 is oriented so that the proximal surface 411 has a smaller diameter than the distal surface 412. As will be discussed more in detail below, the interfacing component 200, in turn, is correspondingly configured to receive the generally disc-shaped body 410 and establish a friction lock with the frustoconical side surface 413. The interfacing component 200 is also configured to securely engage the polymer glenosphere 100 by a combination of a longitudinally-hollow tightening screw 250 and a press-fitting engagement. This arrangement will be described in more detail below.

Referring to FIGS. 1, 4, and 8B, the interfacing component 200 is a generally bowl-shaped structure having a convex side 201 and a concave side 202. The convex side 201 can be configured to be received in the circular recessed portion 120 of the polymer glenosphere 100 and securely engage the glenosphere 100 by press-fitting into the recessed portion 120 of the polymer glenosphere 100. The convex side 201 comprises a side surface 220 that is configured to engage the circular recessed portion 120 of the polymer glenosphere 100 to establish the press-fit engagement.

The concave side 202 of the interfacing component 200 can be configured with an annular female-type tapered surface 202A to receive the disc-shaped body 410 of the baseplate 400 and form a secure attachment by cooperation of the annular female-type tapered surface 202A with the tapered side surface 413 of the baseplate 400 to form a friction lock engagement. The taper on the annular tapered surface 202A and the tapered side surface 413 can be Morse tapers.

The interfacing component 200 can be made of a material that is more rigid than the polymer glenosphere 100 to provide a desired structural rigidity when the interfacing component 200 is assembled with the polymer glenosphere 100. More rigid here means that the interfacing component 200 can be made of a material having Young's modulus of at least 720 Mpa. The convex side 201 of the interfacing component 200 can have a contour that generally follows the interior contour of the circular recessed portion 120 of the polymer glenosphere 100 and form an interface between the interfacing component 200 and the polymer glenosphere 100.

Preferably, the convex side 201 of the interfacing component 200 and the interior of the circular recessed portion 120 can form intimate contact along some portions of the interface to establish the press-fit engagement. For example, the convex side 201 can be configured so that its side surface 220 along its periphery is a male-type tapered surface that forms an intimate contact with the annular interior surface 125 of the recessed portion 120 of the polymer glenosphere 100 and form press-fit between the interfacing component 200 and the polymer glenosphere 100. To accomplish this, the annular interior surface 125 can be configured as a corresponding female-type tapered surface.

FIG. 8A is a lateral view of a partial assembly 10A in which the polymer glenosphere 100 has been assembled with the interfacing component 200, the tightening nut 250, and the securing screw 300. Referring to FIG. 8B, which is a longitudinal sectional view of the partial assembly 10A of FIG. 8A taken through the line B-B shown in FIG. 8A, the assembly 10A is radially symmetric about the assembly's longitudinal axis 15A. When the polymer glenosphere 100 and the interfacing component 200 are assembled, the interfacing component 200 provides a rigid frame for the polymer glenosphere.

Referring to FIG. 1, when the polymer glenosphere 100, the interfacing component 200, the tightening nut 250, the securing screw 300, and the baseplate 400 are assembled, the resulting glenoid implant assembly 10 is radially symmetric about the glenoid implant assembly's longitudinal axis 15. The longitudinal axis 15A of the partial assembly 10A coincides with the glenoid implant assembly's longitudinal axis 15. In the glenoid implant assembly 10, the interfacing component 200 is interposed between the polymer glenosphere 100 and the baseplate 400 and provide a secure attachment between the polymer glenosphere 100 and the baseplate 400.

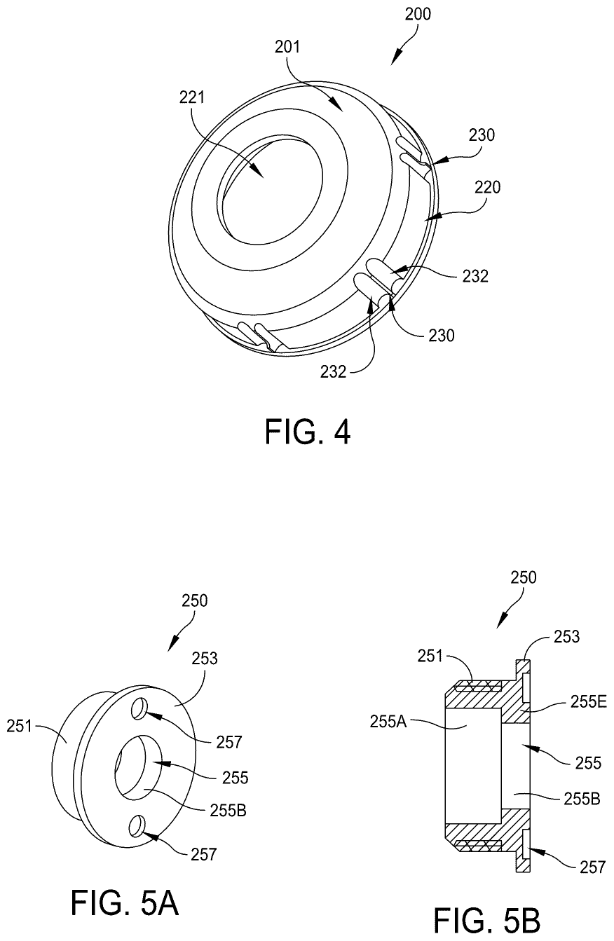

Referring to FIGS. 2 and 4, the circular recessed portion 120 of the polymer glenosphere 100 comprises an annular interior surface 125 along its periphery that is configured to engage the side surface 220 of the convex side 201 of the interfacing component 200 to establish the press-fit engagement mentioned above. The outer diameter of the convex side 201 of the interfacing component 200 and the inner diameter of the annular interior surface 125 of the polymer glenosphere 100 are sized to effectively form the press-fit. In other words, the outer diameter of the convex side 201 can be incrementally larger than the inner diameter of the annular interior surface 125 resulting in an interference that enables the press-fitting. The degree of this interference can be optimized to form the desired level of press-fitting depending on the overall dimensions of the components and the particular materials selected for the components.

Referring to FIG. 4, in some embodiments, the side surface 220 of the interfacing component 200 can comprise a plurality of anti-rotation blades 230 configured to engage and interfere with the annular interior surface 125 to prevent rotational movement between the polymer glenosphere 100 and the interfacing component 200. The anti-rotation blades 230 radially extend outward beyond the side surface 220 which allows the anti-rotation blades 230 to press into the annular interior surface 125 of the polymer glenosphere 100 when the press-fit engagement is made.

Referring to FIGS. 2, 4, 5A, 5B, and 8B, in some embodiments, the polymer glenosphere 100 and the interfacing component 200 each comprises an aperture 131, 221, respectively, extending through the polymer glenosphere 100 and the interfacing component 200, respectively, along the glenoid implant assembly's longitudinal axis 15. The glenoid implant assembly 10 further includes a longitudinally-hollow tightening screw 250 that is inserted through the aperture 221 in the interfacing component from the concave side 202 and threading into the aperture 131 in the polymer glenosphere to establish a second attachment mechanism, in addition to the press-fitting engagement described above. The tightening nut 250 is configured to capture the interfacing component 200 between the tightening nut 250 and the polymer glenosphere 100.

Referring to FIG. 2, the aperture 131 in the polymer glenosphere 100 expands stepwise into two portions 132 and 133 having successively larger diameters starting from the initial aperture 131. The initial aperture 131 will be referred to as the first portion. The larger diameter portion 132 will be referred to as the second portion. The largest diameter portion 133 will be referred to as the third portion. The third portion 133 is threaded to receive the tightening nut 250, and the second portion 132 is threaded to receive the threaded head 310 of the securing screw 300.

Referring to FIGS. 5A-5B, in some embodiments, the tightening nut 250 can be configured with a radially extending flange 253 that captures the interfacing component 200 between the tightening nut and the polymer glenosphere in the glenoid implant assembly 10. The tightening nut 250 comprises an externally threaded portion 251 that has the appropriate outer diameter to thread into the third portion 133 of the aperture 131 in the polymer glenosphere 100. The tightening nut 250 can be configured to accommodate the use of an instrument to assist in threading and tightening the tightening nut 250 into the third portion 133. For example, instrument receiving features such as the two or more recesses 257 shown can be provided on the distal surface of the tightening nut 250. An appropriate instrument provided with matching number of prongs can be used to engage the two or more recesses 257 to turn the tightening nut 250.

Referring to FIGS. 1, 5A-5B, and 8B, the tightening nut 250 also comprises a central aperture 255 that extends longitudinally through the whole length of the tightening nut 250. The central aperture 255 coaxially aligns with the apertures 131, 221 in the polymer glenosphere and the interfacing component, respectively, along the longitudinal axis 15 of the glenoid implant assembly 10 to allow a securing screw 300 (discussed in more detail below) to extend through the aperture 255 in the tightening nut 250. The central aperture 255 includes two portions: a smaller diameter first portion 255B and a larger diameter second portion 255A that is proximally positioned with respect to the first portion 255B. The diameter of the first portion 255B is sized to allow the threaded portion 322 of the securing screw 300 to slide through. The proximal direction referred to herein coincides with the proximal and distal directions identified for the glenoid implant assembly 10.

Referring to FIGS. 1 and 3, in some embodiments, the glenoid implant assembly further comprises a securing screw 300 comprising a threaded screw head 310 and a shaft 320. The shaft 320 comprises a threaded portion 322 that is adjacent to the threaded head 310 and a non-threaded portion 323 extending distally and away from the threaded head 310. The threaded screw head 310 has threaded surface 312 that threads into the second portion 132 of the aperture 131 in the polymer glenosphere 100 from the recessed portion 120 of the polymer glenosphere with the shaft 320 extending distally. The screw head 310 can be provided with a tool-receiving recess 315 so that an appropriate screw driver tool can be used to turn the screw 300 as necessary.

Referring to FIGS. 9A-9B, in some embodiments, the baseplate 400 comprises a centrally located aperture 425 extending from the proximal surface 411 through the length of the central stem 420. The aperture 425 is configured to receive the non-threaded portion 323 of the shaft 320 of the securing screw 300. The interior surface of the aperture 425 and the surface of the non-threaded portion 323 can be finished with an appropriate surface finish so that they interact with each other smoothly with minimum friction.

When the partial assembly 10A shown in FIGS. 8A-8B is being secured to the baseplate 400, the shaft 320 of the securing screw 300 extending distally beyond the distal surface 112 of the glenosphere 100 is inserted into the aperture 425 and acts as a stabilizing guide and assist in aligning the circular recessed portion 120 of the polymer glenosphere 100 with the baseplate 400 so that the complementary tapered surfaces 125 (on the glenosphere) and 413 (on the baseplate) are properly engaged.

Referring again to FIGS. 9A-9B, in some embodiments, the baseplate 400 comprises one or more apertures 427 extending through the baseplate 400 adjacent to the outer periphery for accommodating anchoring members such as bone screws 500. The bone screws 500 are used to secure the baseplate 400 to the glenoid. Referring to FIG. 9A, for each of the one or more apertures 427, the opening on the proximal surface 411 of the baseplate is configured with a shoulder or a countersink 427C to accommodate the head of the bone screw 500. The countersink 427A has a curved surface and otherwise configured to accommodate the head of the bone screw 500 in a variety of screw angles. Referring to FIG. 9B, for the same reason, each of the openings for the apertures 427 on the distal surface 412 of the baseplate is configured with a shoulder 427B having a frustoconical surface to accommodate the shaft of the bone screw 500 in a variety of screw angles.

In some embodiments, one or more of the apertures 427 for the bone screws 500 can be provided with an internal member 440 that is disposed within the aperture 427. When an internal member 440 is provided, the internal member 440 can be semi-spherical and the aperture holding the internal member 440 is also semi-spherical to permit movement of the internal member 440 with respect to the baseplate 400. The movement of the internal member 440 can rotation and/or tilting. Each internal member 440 has a threaded hole as shown for receiving a bone screw 500. The movable feature of the internal member 440 allows a bone screw received therein to be aimed along a desired vector.

### Preparing the Glenoid and Securing the Baseplate:

The surgical procedure for implanting the glenoid implant assembly 10 of the present disclosure would include at least the following steps. First, the glenoid surface is prepared to receive the baseplate 400 by reaming the glenoid surface as necessary to receive and meet the distal surface 412 of the baseplate 400. Then, an appropriately sized hole is drilled into the glenoid surface for receiving the central stem 420 of the baseplate 400. The baseplate 400 is then seated onto the prepared glenoid surface. Next, one or more holes are drilled into the glenoid using the one or more apertures 427 in the baseplate 400 as guides. Then, bone screws 500 are used to secure the baseplate 400 to the glenoid.

### Assembling the Glenosphere, Interface Component, and the Securing Screw:

Either before or after the glenoid is prepared, the partial assembly 10A shown in FIGS. 8A-8B can be assembled. First, the securing screw 300 is secured to the polymer glenosphere 100 from the circular recessed portion 120 side of the glenosphere 100. The threaded portion 312 of the threaded head 310 of the securing screw is threaded into the second portion 132 of the aperture 131 of the glenosphere 100 with the securing screw oriented so that the shaft 320 is pointing in the distal direction and extending out of the circular recessed portion 120 as shown.

Next, the interfacing component 200 and the polymer glenosphere 100 is assembled by inserting the convex side 201 of the interfacing component 200 into the circular recessed portion 120 of the glenosphere 100. Because the circular recessed portion 120 and the convex side 201 are dimensioned for press-fitting, the interfacing component 200 may need to be pushed into the circular recessed portion 120 with some force.

Once the interfacing component 200 is seated inside the circular recessed portion 120 of the polymer glenosphere 100, the securing screw's shaft 320 is extending through the aperture 221 of the interfacing component 200 and positioned in the center of the aperture 221.

Next, the tightening nut 250 is used to further secure the interfacing component 200 and the glenosphere 100. With the tightening nut 250 oriented with the end having the flange 253 distally of the polymer glenosphere 100, the tightening nut 250 is slipped over the shaft 320 of the securing screw 300 and advanced proximally through the aperture 221 in the interfacing component 200 until the externally threaded portion 251 engages the threaded third portion 133 of the polymer glenosphere 100. Then, the tightening nut 250 is turned and screwed into the polymer glenosphere 100 until the flange 253 captures the annular edge of the aperture 221 of the interfacing component 200 between the flange 252 and the polymer glenosphere 100. The tightening nut 250 is screwed into the glenosphere 100 until desired torque is reached.

FIG. 8B shows the configuration of the partial assembly 10A once the tightening nut 250 is screwed in and tightened to the desired level.

### Assembling the Partial Assembly 10A to the Baseplate:

The completed partial assembly 10A is then secured to the baseplate 400 that has been installed onto a glenoid by inserting the shaft 320 of the securing screw 300 into the aperture 425 of the baseplate 400 until the configuration shown in FIG. 1 is achieved. During this insertion, the female-type tapered surface 202A of the interfacing component 200 and the male-type tapered surface 413 of the baseplate 400 engage and form a friction-lock engagement.

After the configuration shown in FIG. 1 is reached, the securing screw 300 is dissociated from the polymer glenosphere 100 by unscrewing the securing screw 300 from the polymer glenosphere 100. This can be achieved by accessing the screw head 310 through the aperture 131 in the glenosphere. An appropriate screw driver tool can be used to engage the tool-receiving recess 315 in the screw head. By unscrewing the screw 300 from the polymer glenosphere 100, the securing screw 300 is dissociated from the polymer glenosphere 100 and advance distally further into the aperture 425 of the baseplate 400 until the threaded portion 322 engages the internal thread of the aperture 425. Then, the securing screw 300 is screwed further into the aperture 425 until the screw head 310 advances distally enough to be within the space S (see FIG. 8C) inside the tightening nut 250 and contact the peripheral edge 255E of the aperture 255 of the tightening nut 250. At this point, screwing the securing screw 300 further into the aperture 425 of the baseplate 400 will cause the screw head 310 to urge the tightening nut 250 and in turn the whole partial assembly 10A (assembly of the glenosphere 100, the interfacing component 200, and the tightening nut 250) toward the baseplate 400 and establish secure engagement between the partial assembly 10A and the baseplate 400.

Although the devices, kits, systems, and methods have been described in terms of exemplary embodiments, they are not limited thereto. Rather, the appended claims should be construed broadly, to include other variants and embodiments of the devices, kits, systems, and methods, which may be made by those skilled in the art without departing from the scope and range of equivalents of the devices, kits, systems, and methods.

We claim:

1. A glenoid implant assembly comprising:
a baseplate comprising a proximal end, a distal end, an outer periphery, and a frustoconical side surface extending from the proximal end to the distal end and defining a male-type tapered surface and configured to be secured to a glenoid;
a polymer glenosphere having a proximal end and a distal end, including a convex articular surface extending distally from the proximal end and a distal surface provided on the distal end, and a circular recessed portion recessed from the distal surface and forming an interior contour of the circular recessed portion; and
an interfacing component, made of a material that is more rigid than the polymer glenosphere, having a bowl-shaped configuration with a convex side and a recessed side, wherein the convex side is configured to secure to the circular recessed portion of the polymer glenosphere;
wherein the recessed side of the interfacing component is configured with an annular interior surface defining a female-type tapered surface to receive and form a friction lock engagement with the male-type tapered surface of the baseplate, and
wherein when the polymer glenosphere, the interfacing component, and the baseplate are assembled into the glenoid implant assembly, the interfacing component is interposed between the polymer glenosphere and the baseplate and provide a secure attachment between the polymer glenosphere and the baseplate.

2. The glenoid implant assembly of claim 1, wherein the convex side of the interfacing component has a contour that generally follows the interior contour of the circular recessed portion of the polymer glenosphere.

3. The glenoid implant assembly of claim 2, wherein the convex side of the interfacing component is configured with a side surface along its periphery that forms an intimate contact along the annular interior surface of the recessed portion of the polymer glenosphere and form press-fit between the interfacing component and the polymer glenosphere.

4. The glenoid implant assembly of claim 1, wherein the polymer glenosphere is made of ultra-high-molecular-weight polyethylene (UHMWPE).

5. The glenoid implant assembly of claim 1, wherein the circular recessed portion of the polymer glenosphere comprises an annular interior surface along its periphery and the convex side of the interfacing component comprises a matching surface comprising a plurality of anti-rotation blades configured to engage the annular interior surface of the circular recessed portion of the polymer glenosphere to prevent rotational movement between the polymer glenosphere and the interfacing component.

6. The glenoid implant assembly of claim 1, wherein the polymer glenosphere and the interfacing component each comprises an aperture extending through along the glenoid implant assembly's longitudinal axis, and the interfacing component and the polymer glenosphere are configured to secure to each other by a tightening nut that is inserted through the aperture in the interfacing component from the recessed side and threading into the aperture in the polymer glenosphere, and also by press-fitting along periphery of the circular recessed portion of the polymer glenosphere, wherein the tightening nut is configured to capture the interfacing component between the tightening nut and the polymer glenosphere.

7. The glenoid implant assembly of claim 6, wherein the circular recessed portion of the polymer glenosphere comprises an annular interior surface along its periphery and the convex side of the interfacing component comprises a matching surface comprising a plurality of anti-rotation blades configured to engage the annular interior surface of the circular recessed portion of the polymer glenosphere to prevent rotational movement between the polymer glenosphere and the interfacing component.

8. The glenoid implant assembly of claim 6, wherein the tightening nut is configured with a radially extending flange that captures the interfacing component between the tightening nut and the polymer glenosphere.

9. The glenoid implant assembly of claim 6, further comprising a securing screw comprising a threaded head and a shaft, wherein the shaft comprises a threaded portion that is adjacent to the threaded head and a non-threaded portion extending away from the threaded head portion,
wherein the threaded screw head threads into the aperture in the polymer glenosphere from the recessed portion of the polymer glenosphere with the shaft extending distally,
wherein the tightening nut has an aperture, extending through the tightening nut, that coaxially aligns with the apertures in the polymer glenosphere and the interfacing component along the longitudinal axis of the glenoid implant assembly to allow the securing screw to extend through the aperture in the tightening nut.

10. The glenoid implant assembly of claim 9, wherein the aperture in the polymer glenosphere comprises a first portion, a second portion, and a third portion starting from the proximal end, wherein the three portions have diameters that are successively larger starting from the first portion, wherein the third portion is threaded to receive the tightening nut, and the second portion is threaded to receive the threaded head of the securing screw.

11. The glenoid implant assembly of claim 9, wherein the baseplate comprises an aperture configured to receive the non-threaded portion of the shaft of the securing screw.

12. The glenoid implant assembly of claim 11, wherein the baseplate comprises one or more apertures extending through the baseplate adjacent to the outer periphery for accommodating bone screws.

13. A glenoid implant assembly comprising:
a polymer glenosphere having a proximal end and a distal end, including a convex articular surface extending distally from the proximal end and a distal surface provided on the distal end, and a circular recessed portion recessed from the distal surface and forming an interior contour of the circular recessed portion; and
an interfacing component, made of a material that is more rigid than the polymer glenosphere, having a bowl-shaped configuration with a convex side and a recessed side, wherein the convex side is configured to secure to the circular recessed portion of the polymer glenosphere;
wherein the recessed side of the interfacing component is configured with an annular interior surface defining a female-type tapered surface to receive and form a friction lock engagement with the male-type tapered surface of the baseplate, and
wherein when the polymer glenosphere and the interfacing component are assembled, the interfacing component provides a rigid frame for the polymer glenosphere.

14. The glenoid implant assembly of claim 13, wherein the convex side of the interfacing component has a contour that generally follows the interior contour of the circular recessed portion of the polymer glenosphere.

15. The glenoid implant assembly of claim 13, wherein the polymer glenosphere is made of ultra-high-molecular-weight polyethylene (UHMWPE).

16. The glenoid implant assembly of claim 13, wherein the circular recessed portion of the polymer glenosphere comprises an annular interior surface along its periphery and the convex side of the interfacing component comprises a matching surface comprising a plurality of anti-rotation blades configured to engage the annular interior surface of the circular recessed portion of the polymer glenosphere to prevent rotational movement between the polymer glenosphere and the interfacing component.

17. The glenoid implant assembly of claim 13, wherein the polymer glenosphere and the interfacing component each comprises an aperture extending through along the glenoid implant assembly's longitudinal axis, and the interfacing component and the polymer glenosphere are configured to secure to each other by a tightening nut that is inserted through the aperture in the interfacing component from the recessed side and threading into the aperture in the polymer glenosphere, and also by press-fitting along periphery of the circular recessed portion of the polymer glenosphere, wherein the tightening nut is configured to capture the interfacing component between the tightening nut and the polymer glenosphere.

18. The glenoid implant assembly of claim 17, wherein the circular recessed portion of the polymer glenosphere comprises an annular interior surface along its periphery and the convex side of the interfacing component comprises a matching surface comprising a plurality of anti-rotation blades configured to engage the annular interior surface of the circular recessed portion of the polymer glenosphere to prevent rotational movement between the polymer glenosphere and the interfacing component.

19. The glenoid implant assembly of claim 17, wherein the tightening nut is configured with a radially extending flange that captures the interfacing component between the tightening nut and the polymer glenosphere.

20. The glenoid implant assembly of claim 17, further comprising a securing screw comprising a threaded head and a shaft, wherein the shaft comprises a threaded portion that is adjacent to the threaded head and a non-threaded portion extending away from the threaded head portion,
wherein the threaded screw head threads into the aperture in the polymer glenosphere from the recessed portion of the polymer glenosphere with the shaft extending distally,
wherein the tightening nut has a aperture, extending through the tightening nut, that coaxially aligns with the apertures in the polymer glenosphere and the interfacing component along the longitudinal axis of the glenoid implant assembly to allow the securing screw to extend through the aperture in the tightening nut.

21. The glenoid implant assembly of claim 17, wherein the aperture in the polymer glenosphere comprises a first portion, a second portion, and a third portion starting from the proximal end, wherein the three portions have diameters that are successively larger starting from the first portion, wherein the third portion is threaded to receive the tightening nut, and the second portion is threaded to receive the threaded head of the securing screw.
